(19) United States
(12) Patent Application Publication
(10) Pub. No.: US 2010/0222711 A1
(43) Pub. Date: Sep. 2, 2010

(54) DEVICES, SYSTEMS AND METHODS FOR CAPTURING BIOMECHANICAL MOTION

(75) Inventor: Darryl LAJEUNESSE, Red Deer (CA)

Correspondence Address: WILSON, SONSINI, GOODRICH & ROSATI, 650 PAGE MILL ROAD, PALO ALTO, CA, 94304-1050, US

(73) Assignee: Sherlock NMD, LLC, a Nevada Corporation

(21) Appl. No.: 12/713,162

(22) Filed: Feb. 25, 2010

Related U.S. Application Data
(60) Provisional application No. 61/155,469, filed on Feb. 25, 2009.
(60) Provisional application No. 61/155,462, filed on Feb. 25, 2009.
(60) Provisional application No. 61/155,456, filed on Feb. 25, 2009.

(51) Int. Cl. A61B 5/11

(52) U.S. Cl. 600/595

(57) ABSTRACT

Systems, devices and methods for capturing motion from a body as disclosed. The systems, devices and methods allow for accurate placement of sensors relative to a body in order capture and analyze motion information.

### CROSS REFERENCE

This application claims the benefit of priority to U.S. Provisional Patent Applications 61/155,469, filed Feb. 25, 2009 and entitled “DEVICES, SYSTEMS AND METHODS FOR CAPTURING BIOMECHANICAL MOTION”; 61/155,462, filed Feb. 25, 2009 and “DEVICES, SYSTEMS AND METHODS FOR ANALYZING ENVELOPES OF FUNCTION”; 61/155,456, filed Feb. 25, 2009 and “DEVICES, SYSTEMS AND METHODS FOR MAINTAINING STRUCTURAL POSITION OF A SUBJECT”; all of which applications are incorporated herein by reference in their entirety.

### BACKGROUND OF THE INVENTION

Musculoskeletal conditions affect one in four adults worldwide and account for a quarter of the total cost of worldwide illness. These conditions are the most common causes of severe long-term pain and physical disability. In the United States alone, more than 1 in 4 people has a musculoskeletal condition requiring medical attention and annual direct and indirect costs for bone and joint health are a staggering $849 billion.

Health care providers rely on an understanding of joint anatomy and mechanics when evaluating a subject's suspected joint problem and/or biomechanical performance issue. Understanding anatomy and joint biomechanics assists in the diagnosis and evaluation of a subject for an orthopedic intervention. However, currently available diagnostic tools are limited in the level of detail and analysis that can be achieved. Typically, when treating joint problems, the intention is to address a specific structural or mechanical problem within the joint. For example, a surgeon might prescribe a specific procedure to correct the joint alignment problem, or a physical therapist might prescribe exercises to strengthen a specific tendon or muscle that is responsible for a joint problem, etc.

It follows, therefore, that the extent to which a specific treatable joint defect can be identified and optimally treated directly impacts the success of any treatment protocol. Currently available orthopedic diagnostic methods are capable of detecting a limited number of specific and treatable defects. These techniques include X-Rays, MRI, discography, and physical exams of the patient. These methods have become widely available and broadly adopted into the practice of treating joint problems and addressing joint performance issues. However, currently available diagnostic techniques provide measurement data that is imprecise and often inconclusive which results in an inability to detect many types of pathologies or to accurately assess pathologies that might be considered borderline. As a result, a significant number of patients having joint problems remain undiagnosed and untreated using current techniques, or are misdiagnosed and mistreated due to the poor clinical efficacy of these techniques.

Imaging is the cornerstone of all modern orthopedic diagnostics. The vast majority of diagnostic performance innovations have focused on static images. Static images are a small number of images of a joint structure taken at different points in the joint's range of motion, with the subject remaining still in each position while the image is being captured. Static imaging studies have focused mainly on detecting structural changes to the bones and other internal joint structures. An example of the diagnostic application of static imaging studies is with the detection of spinal disc degeneration by the use of plain X-rays, and MR images. However, these applications yield poor diagnostic performance with an unacceptably high proportion of testing events yielding either inconclusive or false positive/false negative diagnostic results (Lawrence, J. S. (1969) Annals of Rheumatic Diseases 28: 121-37; Waddell, G. (1998) The Back Pain Revolution. Edinburgh, Churchill Livingstone Ch2 p 22; Carragee et al. (2006) Spine 31(5): 505-509, McGregor et al. (1998) J Bone Joint Surg (Br) 80-B: 1009-1013; Fujiwara et al. (2000(a)) Journal of Spinal Disorders 13: 444-50).

A method for determining vertebral body positions using skin markers was developed (Bryant (1989) Spine 14(3): 258-65) but could only measure joint motion at skin positions and could not measure the motion of structures within the joint. There have been many examples of skin marker based spine motion measurement that are similarly challenged.

Methods have been developed to measure changes to the position of vertebrae under different loads in dead subjects, whose removed spines were fused and had markers inserted into the vertebrae (Esses et al. (1996) Spine 21(6): 676-84). The motion of these markers was then measured in the presence of different kinds of loads on the vertebrae. Other methods with living subjects have been able to obtain a high degree of accuracy in measuring the motion of internal joint structures by placing internal markers on the bones of subjects and digitally marking sets of static images (Johnsson et al. (1990) Spine 15: 347-50), a technique known as roentgen stereophotogrammetry analysis (RSA). However RSA requires the surgical implantation of these markers into subjects' internal joint structures, requires the use of two radiographic units simultaneously, and requires a highly complicated calibration process for each test, and therefore is too invasive and too cumbersome a process for practicable clinical application.

Current processes fail to control motion during testing and do not adequately account for the involvement and effects of muscles that are acting when a subject moves under their own muscular force while in a weight-bearing stance. Such movement adds variability by introducing such inherently variable factors such as the subject's muscle strength, level of pain, involuntary contraction of opposing muscle groups, and neuro-muscular co-ordination. Taken together, all of these sources of variability serve to confound diagnostic conclusions based on comparative analyses by making the ranges of “normal” and those of “abnormal” difficult to distinguish in a statistically significant manner. Such inability to distinguish between “normal” and “unhealthy” subjects based on a specific diagnostic measurement renders such a measurement diagnostically impracticable, as has been the case heretofore with methods that have focused on measurements of uncontrolled joint motion measured in subjects in weight-bearing postures and moving their joints through the power of their own muscles and in an uncontrolled fashion.

U.S. Pat. No. 5,505,208 to Toomin et al. developed a method for measuring muscle dysfunction by collecting muscle activity measurements using electrodes in a pattern across a subject's back while having the subject perform a series of poses where measurements are made at static periods within the movement. These electromyographical readings of “unhealthy” subjects were then compared to those of a “normal” population so as to be able to identify those subjects with abnormal readings. However, the technique does not provide a method to report the results as a degree of departure from an ideal reading, and instead can only report whether a reading is “abnormal.” U.S. Pat. No. 6,280,395 to Appel et al. added an additional advantage to this method the ability to better normalize the data by employing a more accurate reading of the thickness of the adipose tissue and other general characteristics that might introduce variability into the readings, as well as the ability to quantify how abnormal a subject's electromyographical reading is as compared to a “normal” population.

What is therefore needed is a system and process for using the system that enables evaluation of human motion and biomechanics.

### SUMMARY OF THE INVENTION

In an aspect, the present invention relates to a 3-dimension scanning system and a 3-dimensional method that enable interpolation to determine movement that can be used to determine general motion capture and physiological mechanics of a body, including the spine and peripheral structures. In another aspect, the present invention relates to devices, systems and methods that are adapted to use a detailed breakdown of functional envelopes (3-dimentional polygons created by analysis of complete biomechanics for the purpose of extrapolating a biomechanical envelope of function (EOF)). In a third aspect, the present invention relates to devices, systems and methods that are adapted to facilitate accurate structural positioning of a mammalian subject.

In an aspect, the invention provides a device for capturing motion from a body comprising a rig adapted to conform to an external shape of the body, wherein the rig comprises two or more elongate members connected by two or more support members. In some embodiments, the device is adapted to conform to at least a portion of a shape of an animal, including without limitation a mammal, human, monkey, primate, horse, cow, dog, cat, rodent, guinea pig, rat or mouse. The device is adapted to conform to a joint, bone or skeletal structure of the body.

In some embodiments, the elongate members comprise a series of telescoping members. The telescoping functionality can comprise a gas charging or liquid charging element.

In some embodiments, the device comprises one or more sensors in communication with the elongate members and/or support members. The sensors can be in electrical communication with the elongate members and/or support members. The sensors can be connected to the elongate members and/or support members via a damped universal joint. Sensors for use with the device include without limitation at least one audio sensor, vibration sensor, or oscillation sensor. Some of the sensors can provide physiological data about the body, whereas other sensors are adaptable to triangulate a plane of the body.

In some embodiments of the device, the support members are connected to the elongate members by an axis ball socket, constant velocity or universal socket system. At least two, three, four, five, six, seven, eight, nine or at least ten elongate members can be provided. In some embodiments, three elongate members are provided. In some embodiments, the device comprises at least two, three, four, five, six, seven, eight, nine, 10, 11, 12, 13, 14, 15, 16, 17, 18, 19 or at least 20 support members. The number of support members can depend on a desired level of accuracy and/or the joint, bone or skeletal structure the device is configured and adapted to conform to.

In some embodiments, the rig is adapted to conform to a spine of the body. In such cases, the rig may comprise three elongate members, e.g., vertical rods, and 5, 6, 7, 8, 9 or 10 support members, e.g., lateral rods. The support members may not be evenly placed along the length elongate members, e.g., the support rods can be placed closer together in the small of the back. In some embodiments, the device comprises a skull rig connected to the spine rig. The device can further comprise a peripheral rig adapted to conform to one or more arms and/or one or more legs of the body. The peripheral rig may be connected to the spine rig or may be separate. The body can wear more than one rig, e.g., on the spine, one or more arms, and/or one or more legs.

In some embodiments, the rig of the device is incorporated into an article of clothing adapted to be worn on the body. The article of clothing can include an organic living exoskeletal morphometry. The article of clothing can be a full or partial body suit, which can include an organic living exoskeletal morphometry and/or a flexible form fitting material. Suitable flexible form fitting materials are known in the art, e.g., those suitable for form fitting athletic wear, including without limitation neoprene, nylon backed neoprene, lycra backed neoprene, cotton, nylon, polyester, elastene, or wool.

In some embodiments, the device is adapted to analyze motion captured from the body using envelopes of function. Markers or sensors can be strategically placed on the rigs of the device to allow detection of the envelopes of function. In some embodiments, the device is adapted to track yaw, pitch and roll via the rig.

In another aspect, the invention provides a system for capturing motion from a body. The system comprises a device for capturing motion as described herein and a computer system configured to capture and/or analyze motion of the body. The system can be adapted to analyze the motion of the body using envelopes of function. The system can be adapted to compare the motion of the body to a model of ideal motion. Such comparison can be used to diagnose a muscular skeletal condition of the body. The comparison can also be used to improve the movement of the body to enhance athletic performance.

In yet another aspect, the invention provides a method for capturing motion from a body. The method comprises providing a device for capturing motion as described herein; conforming one or more rigs of the device to the body; and capturing the motion of the body using the one or more rigs. Conforming the one or more rigs of the device to the body may comprise attaching one or more sensors to triangulated positions relative to bony landmarks in the body and/or structural dead areas of the body. Such placement can facilitate more accurate motion detection. In some embodiments, the motion data from the body is analyzed using envelopes of function. The method can include comparing the motion of the body to a model of ideal motion. The comparison can include comparing envelopes of function of the body to those projected for ideal or improved movement. Such comparisons can be used to diagnose a muscular skeletal condition of the body. Thus, in embodiments, the comparison is used to diagnose a motion disorder or determine the efficacy of a course of treatment for treating a motion disorder. The comparison can also be used to improve the movement of the body to enhance athletic performance.

In a related aspect, the invention provides a method for capturing motion from a body comprising: providing a system that includes a device for capturing motion as described herein and a computer system configured to capture and/or analyze motion of the body, conforming one or more rigs of the device for capturing motion to the body; and capturing the motion of the body using the one or more rigs. Conforming the one or more rigs of the device to the body may comprise attaching one or more sensors to triangulated positions relative to bony landmarks in the body and/or structural dead areas of the body. Such placement can facilitate more accurate motion detection. In some embodiments, the motion data from the body is analyzed using envelopes of function. The method can include comparing the motion of the body to a model of ideal motion. The comparison can include comparing envelopes of function of the body to those projected for ideal or improved movement. Such comparisons can be used to diagnose a muscular skeletal condition of the body. Thus, in embodiments, the comparison is used to diagnose a motion disorder or determine the efficacy of a course of treatment for treating a motion disorder. The comparison can also be used to improve the movement of the body to enhance athletic performance.

In another aspect, the invention provides an adjustable station adapted to capture a sensed parameter from a body. The station comprises a base plate and a support framework protruding from the base plate, wherein the support framework comprises a support rail. The base plate can be substantially flat or another shape that allows the body to stand on the base plate. The base plate can include one or more pressure pads adapted to support a weight of the body. The pressure pads can be configured to be adjustable to accommodate a variety of body sizes. In some embodiments, the pressure pads are adjustable anteriorly and/or posteriorly.

The support rail of the adjustable station can be adapted to be held onto by the body. For example, the hands of the body can be placed on support rail. In some embodiments, the support framework comprises at least one side rail connected to the base plate, and at least one of the at least one side rails support the support rail. The support rail can be substantially perpendicular to the base plate and/or vertically adjustable. In some embodiments, the adjustable station comprises two side rails positioned on or near opposite side edges of the base station, wherein the support rail runs between the side rails and the side rails support the support rail, which is itself positioned over a third edge of the base station at a height that can be held onto by the body while the body is standing on the base plate.

In some embodiments of the adjustable station, the support framework comprises pressure sensors. The pressure sensors can be adapted to sense the pressure exerted by the body on the support rail. In one embodiment, the support rail comprises one or more hand sensors that are adjustable along the length of the support rail. One or more of the one or more side rails can also include a hand sensor that is adjustable along a length of the side rail.

In some embodiments, the adjustable station can be adapted to capture a sensed parameter from a human body in a standing or crouched position. The body can stand on the station and pressure can be sensed from the base station and support framework. The subject can also don a device for motion capture according to the invention while standing on the adjustable station.

In another aspect, the invention provides a system comprising: a device for motion capture as described herein; and an adjustable station as described herein. The system can further include a computer system configured to capture and/or analyze a position or a motion of the body. The system can be adapted to analyze the motion of the body using envelopes of function. The system can be adapted to compare a position or motion of the body to a model position or motion.

In a related aspect, the invention provides a method for calibrating a motion capture device placed on a body comprising: providing a device for motion capture as described herein; providing an adjustable station as described herein. The device for motion capture, e.g., one or more rigs and or a motion capture suit, is conformed to the body and the body is placed on the base plate of the adjustable station, e.g., in a standing position. Optionally, the body can grasp the support rail of the adjustable station. The device for capturing motion is calibrated to the body while the body is positioned in the adjustable station. In some embodiments, the method further comprises providing a computer system configured to capture and/or analyze a position and/or a motion of the body.

In another aspect, the invention provides a method for diagnosing a muscular skeletal condition of a human subject. The method comprises providing a flexible form fitting body suit adaptable to be worn by the subject, wherein the body suit comprises a series of sensors placed on the skull and placed along a length of the arms, legs, spine, and stomach areas of the body suit. The method also comprises providing an adjustable station comprising a base plate comprising two pressure sensing plates adapted to support the weight of the subject; and a support framework protruding from the base plate, wherein the support framework comprises a support rail supported by two side rails, wherein the support rail is adapted to be held by the subject, and wherein the support rail comprises two adjustable hand sensors. The method further comprises providing a computer system configured to capture and/or analyze a position and/or a motion of the subject. According to the method, the subject dons the body suit and is then placed in a standing position within the adjustable station with one foot positioned on one of the two pressure sensing plates, the other foot positioned on the other of the two pressure sensing plates, one hand holding one of the adjustable hand sensors on the support rail, and the other hand holding the other adjustable hand sensor on the support rail. The suit is adjusted while the subject is standing within the adjustable station, wherein the adjusting comprises comparing the position of the user and the sensors on the body suit against a 3D model of the user generated by the computer system; and repositioning the suit and/or calibrating the detection system until the position of the user and the sensors on the body suit meet a desired level of calibration as determined by the computer system. The level of calibration can be that determined to be necessary for medical diagnosis and/or treatment. The method further entails capturing motion of the subject while the subject is wearing the adjusted and calibrated suit and transmitting the capture data to the computer system in real time. The motion of the subject is compared to a model of the same motion generated by the computer system and the comparison in used to diagnose the muscular skeletal condition. In embodiments, the comparison comprises analyzing the motion of the subject using envelopes of function.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 illustrates a spine rig according to an embodiment shown from the front, rear, side, front perspective and rear perspective views;

FIG. 2A illustrates a solid exoskeleton with interleaving of a grasshopper; FIG. 2B illustrates a solid exoskeleton with interleaving of a bee;

FIG. 3 illustrates the exemplary spine rig of FIG. 1 in combination with a human spinal column and skull from the same views;

FIG. 4 illustrates a close-up of a spine rig according to an embodiment shown from a rear perspective, rear, front and front perspective view;

FIG. 5 illustrates a close-up exploded view of a spine rig according to an embodiment shown from the front perspective view (cut), side perspective view (cut), front perspective view (exploded), rear perspective view (exploded), and rear perspective view (cut);

FIG. 6 illustrates a rear perspective cut and exploded views of a spine rig according to an embodiment;

FIG. 7 illustrates the rear detailed view of a spine rig according to an embodiment;

FIGS. 8A-8E illustrate an exemplary motion capture suit. FIG. 8A illustrates a perspective frontal head shot with skull rig. FIG. 8B illustrates a perspective full frontal view of the suit. FIG. 8C illustrates a full frontal view of the suit with cutout showing the underlying musculature. FIG. 8D illustrates a perspective rear headshot with skull rig. FIG. 8E illustrates a perspective full rear view of the suit.

FIG. 9 illustrates a front perspective view showing envelopes of function;

FIG. 10 illustrates a rear perspective view showing the envelopes of function;

FIG. 11 illustrates a top perspective view showing the envelopes of function;

FIG. 12 illustrates a computer system having components suitable for use in the invention;

FIG. 13 illustrates a front and rear perspective view of a motion sensing station according to an embodiment;

FIG. 14 illustrates, front, rear and top views of a motion sensing station according to an embodiment;

FIG. 15 illustrates a human skull from different perspectives having a head rig associated therewith; and

FIG. 16 illustrates a human skeleton with a head rig standing on a motion sensing station according to an embodiment.

### DETAILED DESCRIPTION OF THE INVENTION

Currently, the most widely used diagnostic tools for muscular skeletal injuries are X-rays and MRI's. These are static diagnostic tests, done with the patient standing or lying perfectly still. Although this is necessary for the identification of bone breaks, fractures and muscle tears, these approaches may not be optimal for the diagnosis of mechanical dysfunctions. Muscular skeletal injuries occur while moving. An accurate diagnosis of injuries that occur while moving requires a diagnostic system that analyses movement. The present invention provides a system and methods that can visualize, analyze and provide diagnostic data while the subject is in motion. The system can be used while a subject is undergoing everyday activities such as walking, turning, bending or running, as well as sports related dynamics such as kicking, throwing, batting, jumping and even contact activities. The invention therefore provides real-time diagnostic images of neuromuscular skeletal function and dysfunction of the human body in motion. It displays true anatomical biomechanics by adjusting to the specific measurements and morphology of each and every subject. This data can then provide doctors, team trainers, physical therapists and other medical personnel or caregivers with the information to quantify specific injuries and biomechanical dysfunctions in relation to applied therapeutic and physical therapy protocols.

The system comprises a 3D medically accurate human anatomical data set. Coupled to this 3D anatomical package is rig that can conform to a subject's body to track their motion. In some embodiments, the system comprises a biomechanically engineered suit and sensor system comprising one or more rigs. The biomechanical 3D anatomical data set and sensor system can be linked to a treatment program via an artificial intelligence (A.I.) engine. These components enable the systems of the invention to quantify specific biomechanical ranges of motion (ROM), function and dysfunction.

The results of using the diagnostic system of the invention include:

1. A clear understanding of the problem

2. More accurate information to develop the right therapeutic approach

3. The ability to track the efficacy of the therapy

4. The guidance to create an optimal follow-up program

5. The chance to reach full athletic excellence by understanding true human biomechanics

The systems and methods can provide benefit to subject's with many sorts of muscular skeletal injuries, including more rapid healing of sports related injuries.

The human spinal column is comprised of a series of thirty-three stacked vertebrae divided into five regions. The cervical region includes seven vertebrae, referred to as C1-C7. The thoracic region includes twelve vertebrae, referred to as T1-T12. The lumbar region contains five vertebrae, referred to as L1-L5. The sacral region is comprised of five fused vertebrae, referred to as S1-S5, while the coccygeal region contains four fused vertebrae, referred to as Co1-Co4. In order to understand the configurability, adaptability, and operational aspects of the invention disclosed herein, it is helpful to understand the anatomical references of the body with respect to which the position and operation of the devices, and components thereof, are described. There are three anatomical planes generally used in anatomy to describe the human body and structure within the human body: the axial plane, the sagittal plane and the coronal plane. Additionally, devices and the operation of devices and tools may be better understood with respect to the caudad direction and/or the cephalad direction. Devices and tools can be positioned dorsally (or posteriorly) such that the placement or operation of the device is toward the back or rear of the body. Alternatively, devices can be positioned ventrally (or anteriorly) such that the placement or operation of the device is toward the front of the body. Various embodiments of the devices, systems and tools of the present invention may be configurable and variable with respect to a single anatomical plane or with respect to two or more anatomical planes. For example, a subject or a feature of the device may be described as lying within and having adaptability or operability in relation to a single plane. For example, a device may be positioned in a desired location relative to a sagittal plane and may be moveable between a number of adaptable positions or within a range of positions.

For purposes of illustration, the devices and methods of the invention are described below with reference to the spine of the human body. However, as will be appreciation by those skilled in the art, the devices and methods can be employed to address any effected bone or joint, including, for example, the hip, the knee, the ankle, the wrist, the elbow, and the shoulder. Additionally, the devices and methods can also be employed with any appropriate subject, e.g., an animal, including without limitation a mammal such as a human, monkey, primate, horse, cow, dog, cat, rodent, guinea pig, rat or mouse.

### Motion Capture

The systems and methods of the invention provide physicians, therapists, trainers and other care providers with a tool to facilitate diagnosis and rehabilitation of underlying neuromuscular skeletal imbalances in motion, resulting in the more complete and long-lasting treatment of injuries. The motion capture device of the invention includes a rig adapted to conform to the shape of a body, e.g., that of a human subject. The rig can be adapted to capture motion of different bones, joints or skeletal structure. Current tools include X-ray machines that provide information regarding bone breaks, fractures, or chips and the MRI machine that provides information regarding soft tissue tears in muscles and tendons as well as ligament damage. These systems provide snapshots of an injury at one point in time. In contrast, the systems presented herein capture and analyze motion in real time to provide information about muscular skeletal positioning and alignment, including when the subject is undertaking a wide range of motion.

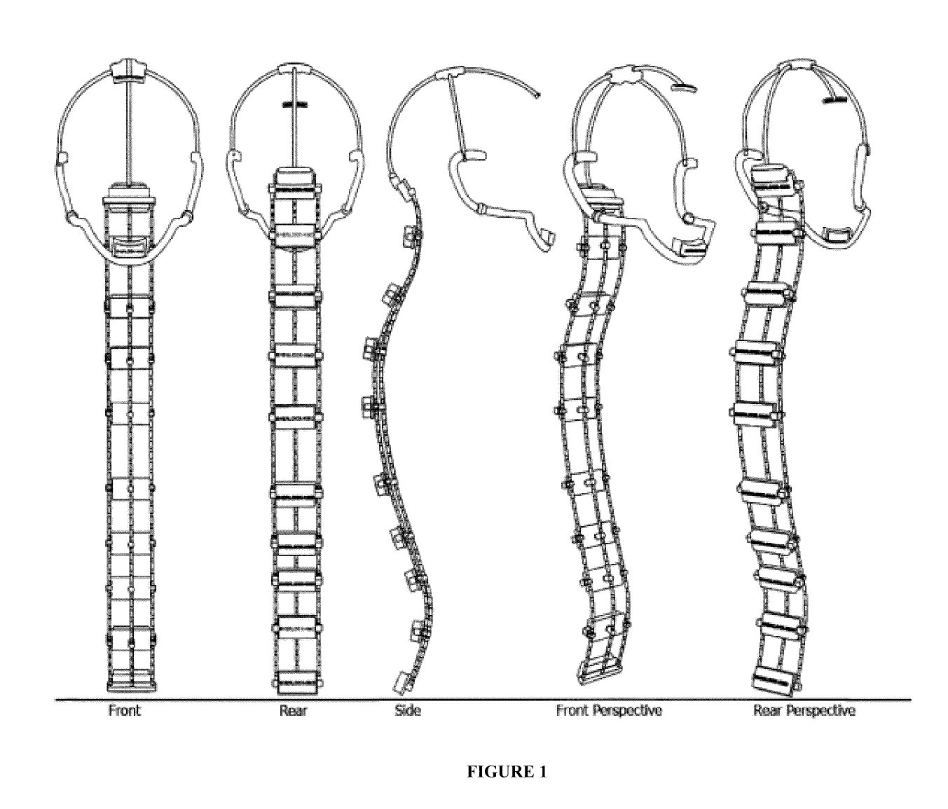

FIG. 1 illustrates a spine rig from the front, rear, side, front perspective and rear perspective views. The system, as depicted here, includes two or more (three depicted) elongate members positioned parallel or substantially parallel to each other which are configured to traverse the length of the skeletal structure, herein a spine. As shown in FIG. 1, the elongate members comprise rods which run vertically to the spine in FIG. 1. The rods may configured such that they are telescoping at a certain distance, e.g., between 5-40 cm. The rods can be telescoping at a distance of 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 31 cm, 32 cm, 33 cm, 34 cm, 35 cm, 36 cm, 37 cm, 38 cm, or 39 cm, typically between 10-30 cm, e.g., 20 cm. The telescoping rods can include an element, e.g., a gas or liquid charging element, to facilitate the telescoping functionality. The telescoping functionality enables a more accurate anatomical fit to a particular subject. As shown in FIG. 1, two or more support members, shown as lateral rods, are also provided connecting the vertical rods at desired locations along its length. The vertical rods can be adapted to be in communication with one or more sensor units positioned in proximity to the spine in order to detect a parameter. In some configurations, lateral connector rods are also connected to or in communication with the sensors. Suitable connection can be via, for example, an axis ball socket system, constant velocity or universal system. In some instances, the center rod, in a three rod configuration, will be connected to one or more sensors, e.g., via a damped universal joint system constant velocity or solid mount using a flexible material. The joint system can be damped to a suitable rate appropriate for a particular application, e.g., a certain pounds per square inch (psi), as will be appreciated by those skilled in the art.

It will also be appreciated that the rig can include at least two, three, four, five, six, seven, eight, nine or at least ten rods, e.g., depending on the particular application or location of the rig, e.g., the size and structure of the joint, bone or skeletal structure being examined. Similarly, depending on the particular application or location of the rig, e.g., the size and structure of the joint, bone or skeletal structure being examined, the rig can include at least two, three, four, five, six, seven, eight, nine, 10, 11, 12, 13, 14, 15, 16, 17, 18, 19 or at least 20 connector rods.

Additionally, sensor placement can be adjusted for a given application of the motion capture device. In some embodiments, the sensors are located via key triangulated positions relative to bony landmarks in the subject's body and key structural dead areas, i.e., locations with superficial bone and little to no soft tissue. Dead areas comprise areas with lack of movement. In those locations, lack of movement relates to the amount of primary and secondary superficial motion. This provides a mechanism for determining areas on the body that are consistent for minimized or anomalous movement or vibration.

Digital audio sensors, similar to those found in digital stethoscopes, can be used with the motion capture device of the invention. Such sensors can be used to monitor muscle baseline contraction, functional intensity, biomechanical endurance, dysfunctional turbulence and action potential/performance. Additionally, sensors can be used that are adapted to sense vibration frequency (e.g. digital audio sensor system), as well as sensors capable of sensing oscillation. Sensors can also be adapted to analyze a sensed parameter.

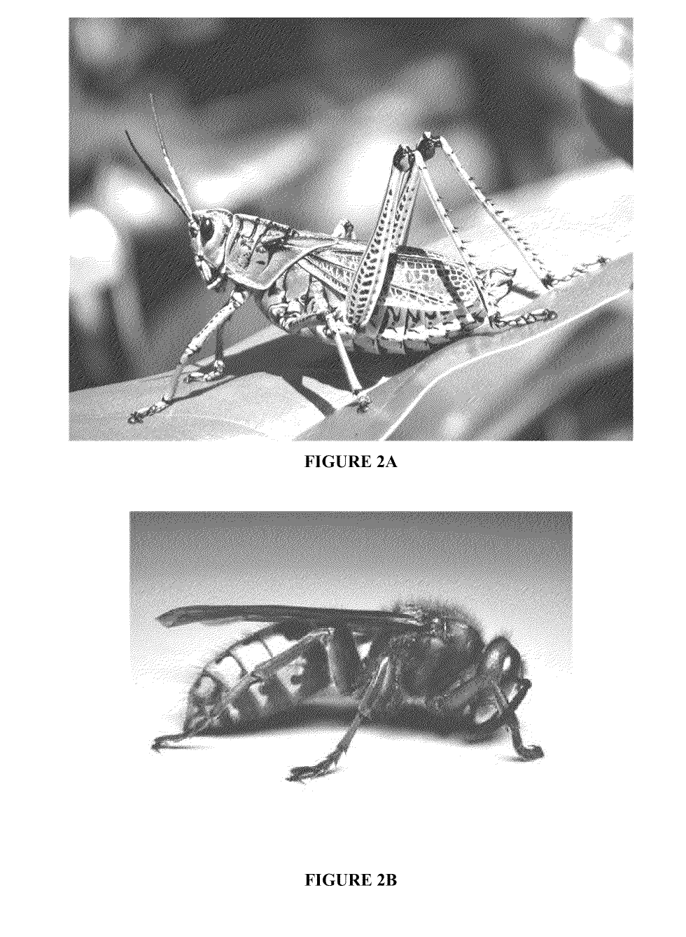

Mechanisms can be provided to ensure that the rig is securely engaging the subject's body. For example, a connector adapted to engage a skull can be provided, as shown in FIG. 1. Other mechanisms can be provided as will be appreciated by those skilled in the art. Additionally, the rig can be incorporated into, for example, an article of clothing, a suit (full or partial body), a jacket, etc., to ensure the rig achieves a relative placement of sensors for a particular individual. The article of clothing can be configured such that it eliminates the need for interpolation and generates accurate biomechanical motion capture for the entire spine of a mammal and/or peripheral appendages. For example, a suit can be configured to capture motion of the spine, skull, one or both arms, and/or one or both legs by having a rig and connectors incorporated in the appropriate positions. The sensor placement allows for micro-rotational movement to be captured (i.e., pitch, roll and yaw), while minimizing and cross-referencing macro translation. The article of clothing can be based on organic living exoskeletal morphometry. For example, many insects use a solid exoskeleton with interleaving. See FIGS. 2A-2B. Such clothing or frame work could employ a similar exoskeletal frame work to achieve organic movement while maintaining structural integrity. The telescoping functionality along the length of the device can further enable the rig to achieve a custom fit to a particular patient in order to optimize data acquisition by the sensors.

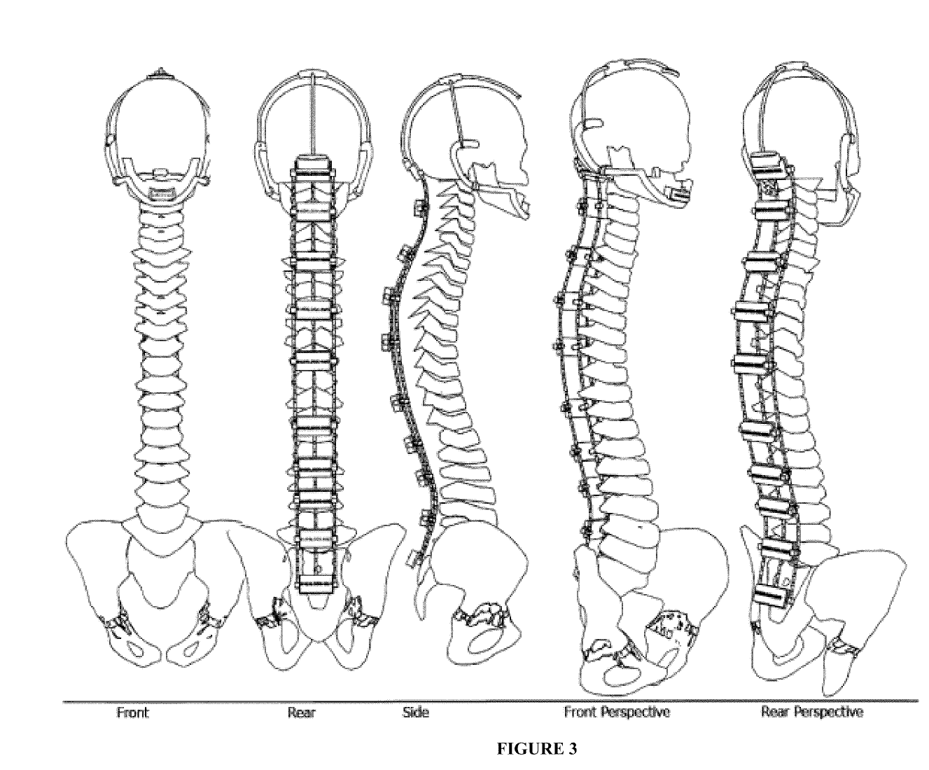

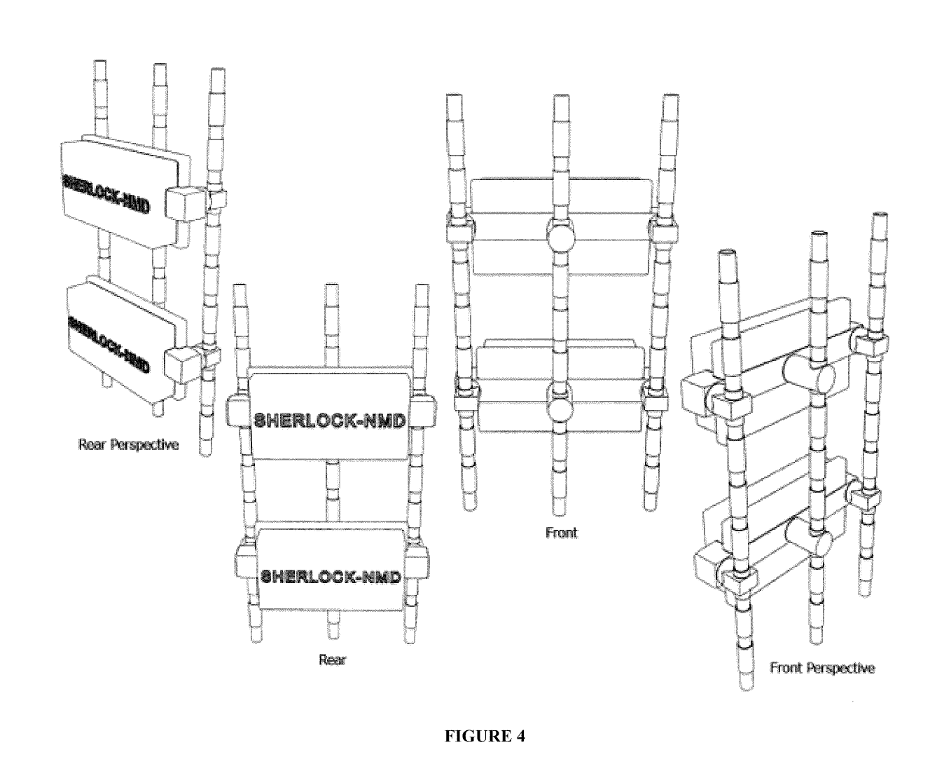

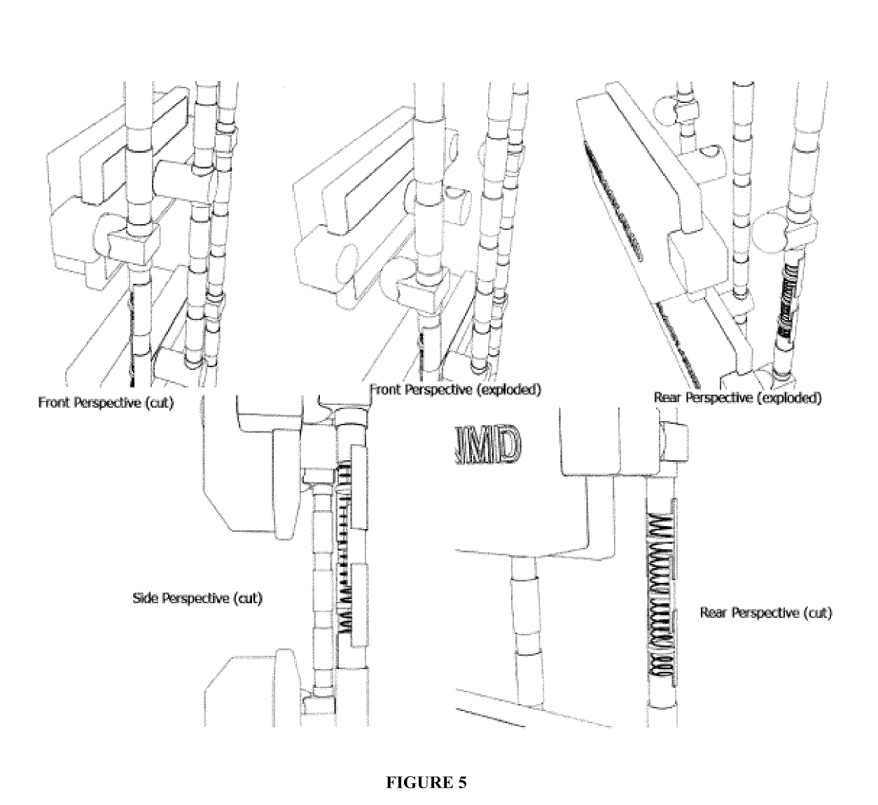

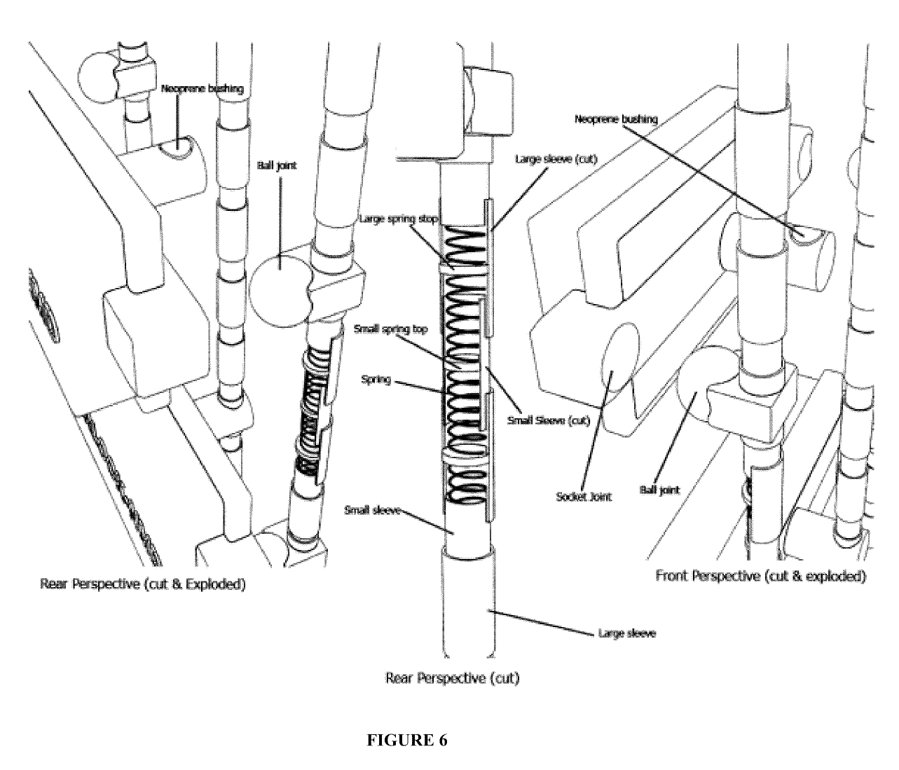

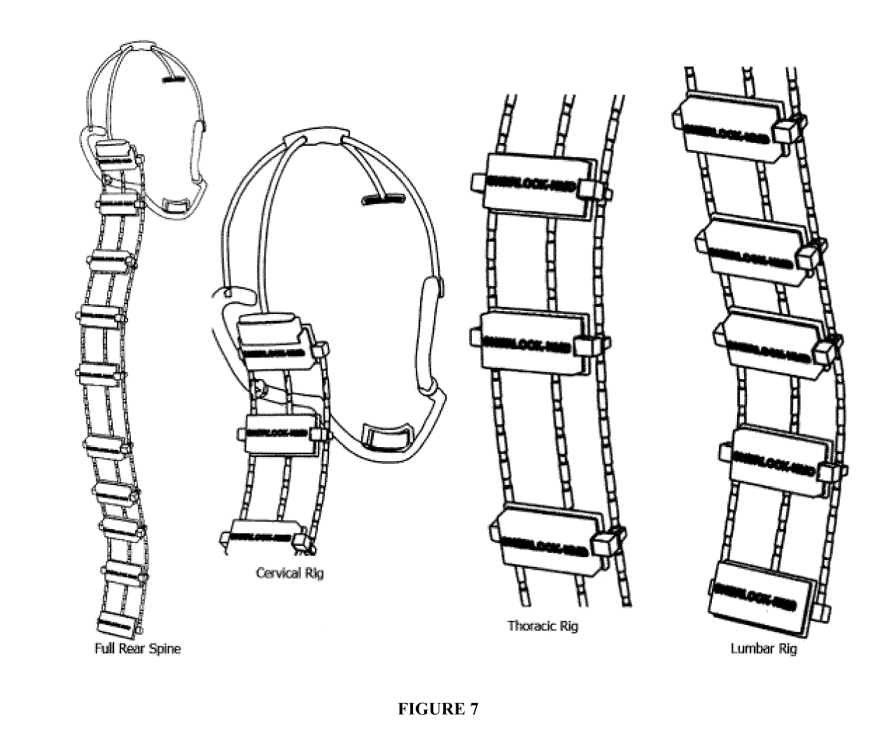

FIG. 3 illustrates the spine rig of FIG. 1 in combination with a human spinal column from the same views. As can be seen, the figure shows the functional relationship with spinal biomechanics and morphology. FIG. 4 illustrates a close-up of the rig from a rear perspective, rear, front and front perspective view. This illustrates a sectional unit in its base form in a structurally neutral orientation. The figure also depicts the sensor array in relation to each other and their specific joint connections to the spinal rig. FIG. 5 illustrates a close-up exploded view of the rig from the from perspective view (cut), side perspective view (cut), front perspective view (exploded), rear perspective view (exploded, and rear perspective view (cut). FIG. 6 illustrates a rear perspective cut and exploded views of the device. This schematic depicts the interleaving nature of the spinal rig with the indication of tension provided by spring. This tension can be provided by, e.g., a gas or liquid charging. It also shows a ball and socket embodiment for connection to the sensor array as well as the central universal connection. FIG. 7 illustrates the rear detailed view of the device showing a macro view of the spinal rig and its functional biomechanical components of the spinal curvatures.

The motion capture systems, devices and methods of the invention can be used quantify the specific effects of therapeutic and/or physical training approaches to in injury detection, prevention, enhancing performance and the treatment of sports injuries and everyday injuries. In some embodiments, the rigs and detection devices of the invention are incorporated into a specifically designed motion capture suit using different types of sensors placed around the joints of the body to provide the user with specific data showing the movement and position of each bone in the body. In some embodiments, at least 2, 3, 4, 5, 6, 7, 8, 9, 10, 15, 20, 25, 30, 35, 40, 45, 50, 65, 70, 75, 80, 85, 90, 95 or at least 100 sensors are placed throughout the suit. In some embodiments, a single sensor could comprise a detector that runs the length of a rig, e.g., an arm, leg, and/or spine. As the number of sensors is increased, a finer granulation of motion capture may be possible.

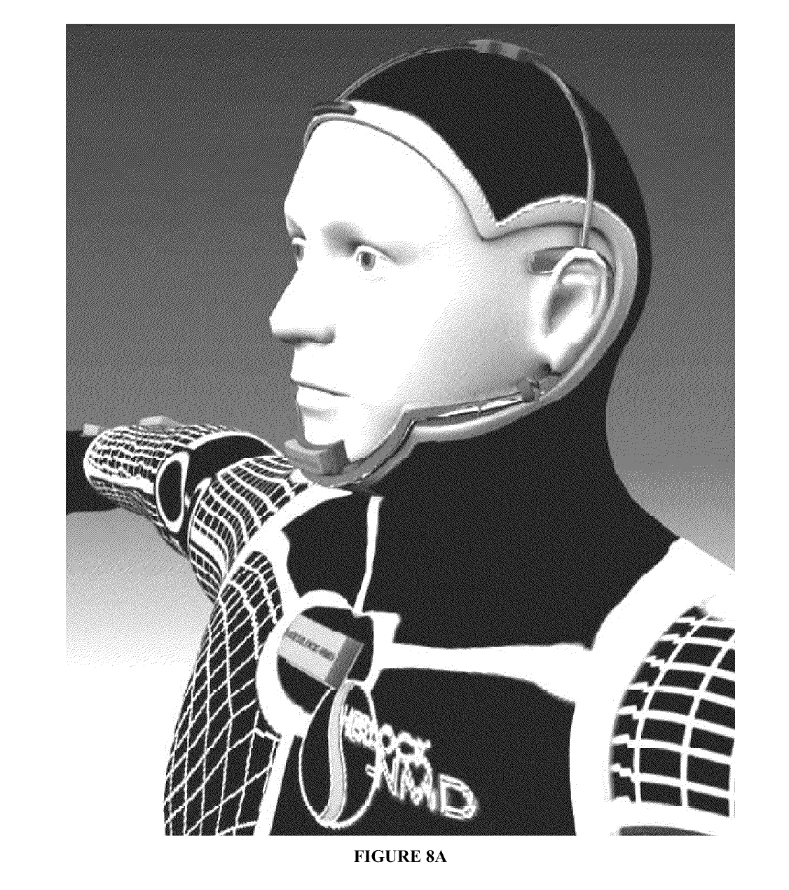

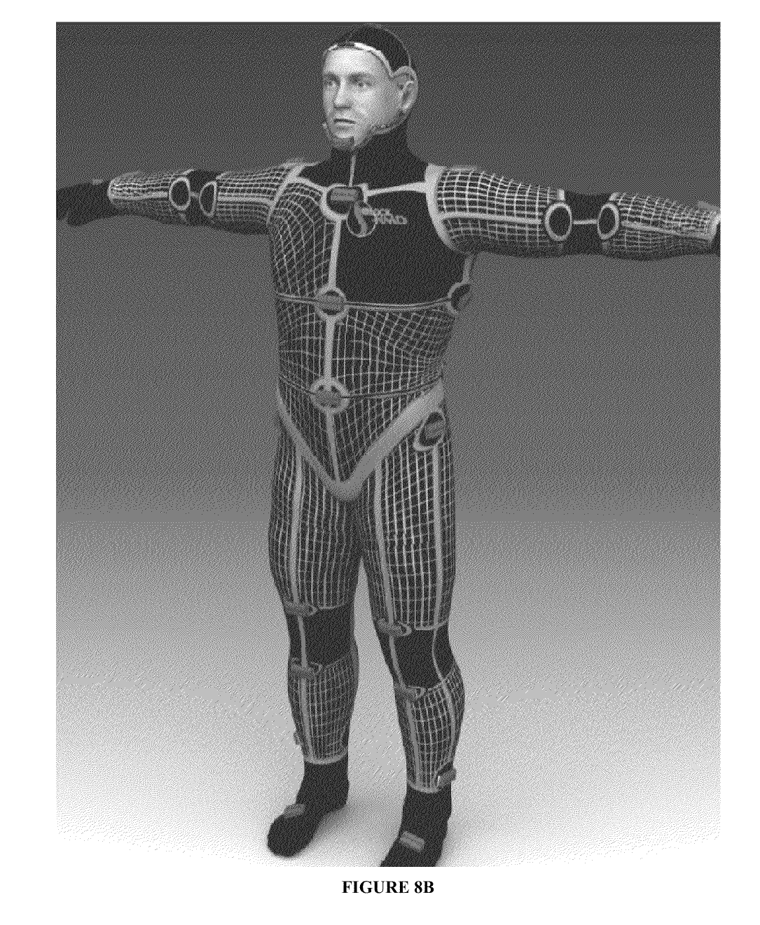

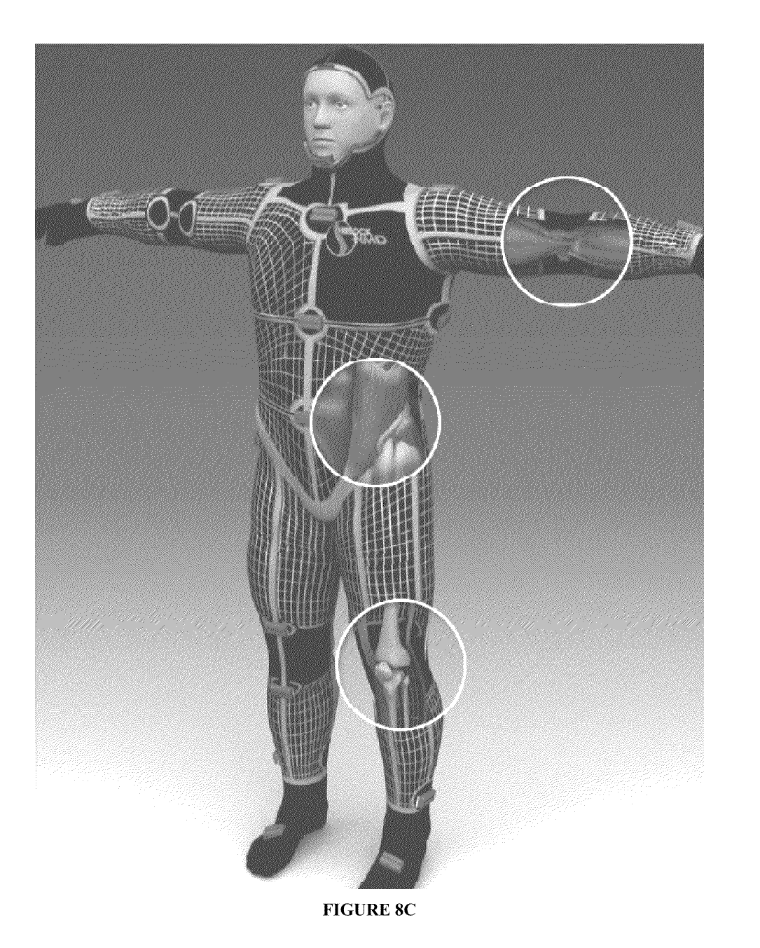

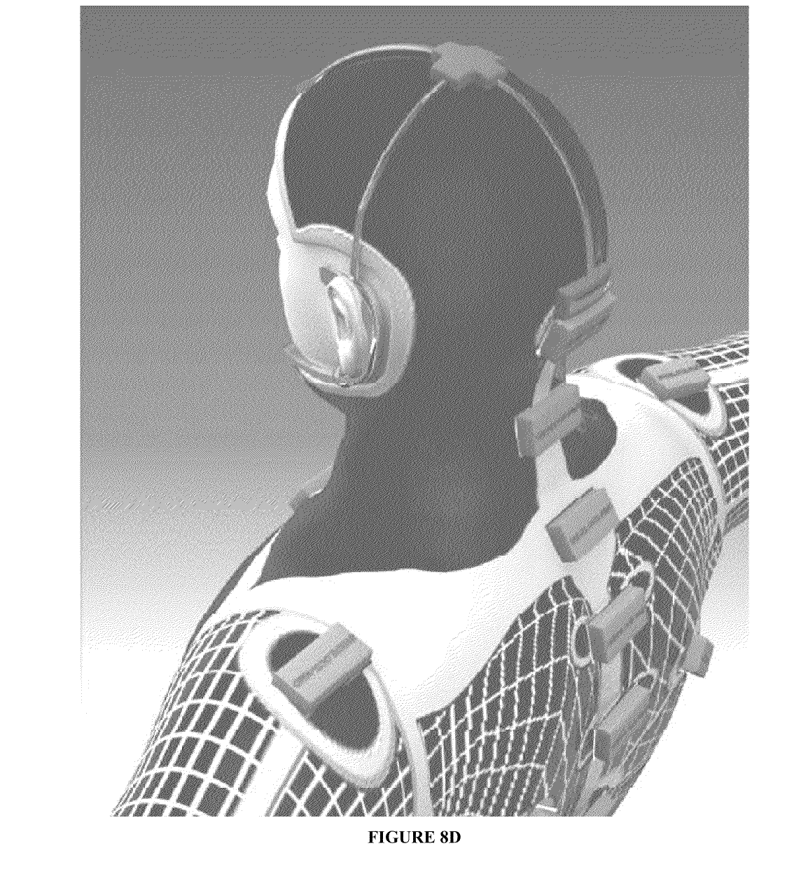

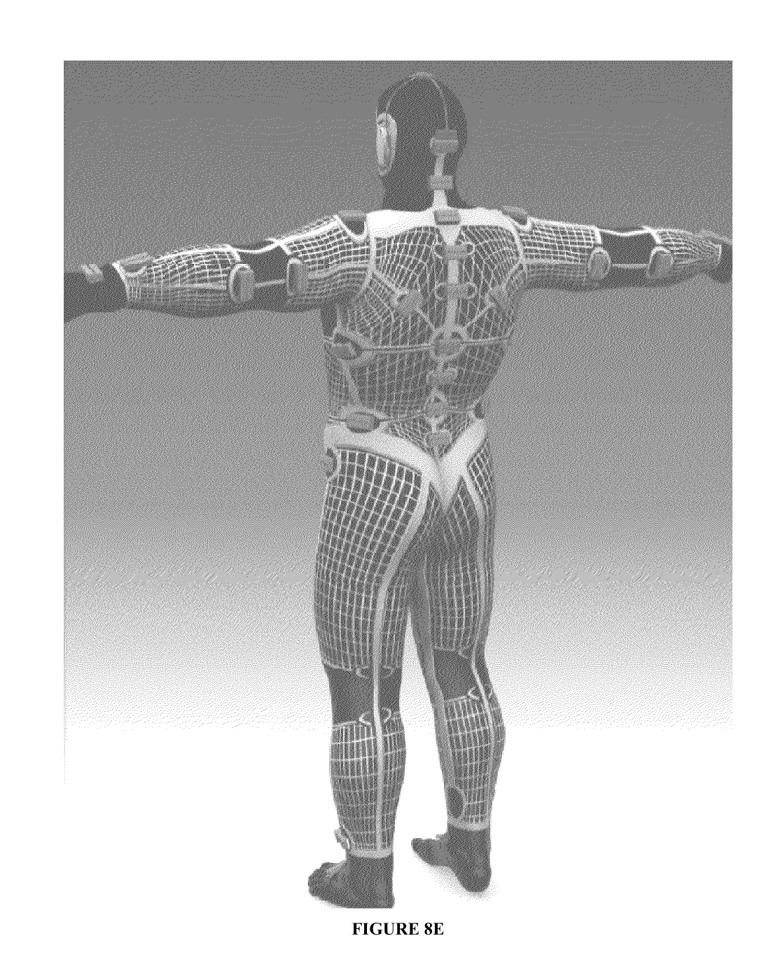

In some embodiments, the rigs and sensors of the invention are incorporated into a body suit. FIGS. 8A-8E illustrate an exemplary motion capture suit. FIG. 8A illustrates a perspective frontal head shot with skull rig. FIG. 8B illustrates a perspective full frontal view of the suit. FIG. 8C illustrates a full frontal view of the suit with cutout showing the underlying musculature. FIG. 8D illustrates a perspective rear headshot with skull rig. FIG. 8E illustrates a perspective full rear view of the suit. The suit can be made in various sizes and have multiple adjustments to accommodate a sufficient fit for subjects of various sizes and shapes. The suit can be made of a comfortable and flexible material to facilitate unencumbered motion by the subject during analysis. In some embodiments, the suit comprises a neoprene material like a foamed neoprene, nylon backed neoprene or lycra backed neoprene. The suit can also comprise cotton, nylon, polyester, elastene, wool, or any other appropriate material such as those used to create clothing, e.g., form fitting athletic wear. The suit can also be formed at least in part using an exoskeletal morphometry. Such configuration may be used wherein the exoskeleton covers on portion of the body, e.g., the chest and/or back, whereas a flexible form fitting material is used for other portions of the suit, e.g., the arms, legs and/or head. As shown in the figures, the suit can comprise a variety of sensors, e.g., those of the spinal and skull rigs of FIGS. 1 and 3-7. Sensors can also be placed on the chest, legs, feet, arms and/or hands. The sensors may be placed on the front, back, and on either side of the suit. In some embodiments, the rigs are incorporated into the suit. In some embodiments, the rigs are deployed external to the suit. One of skill will appreciate that a partial suit can be used as appropriate for a given situation. For example, only the shirt portion may be worn if the shoulder is being monitored, or only the legs may be worn if an ankle or knee is being evaluated. One of skill will understand that a suit or portions thereof can be configured into various configurations such as these or others. The suit can have patterns on the outer surface to facilitate motion detection. Non-limiting exemplary placements are shown throughout FIGS. 8A-8E. The deflection of a pattern during motion provides an indication of the motion of the subject.

One of skill will appreciate that a variety of systems can be used to detect the motion of the rig and/or suit worn by the subject. The rigs and/or suits can have markers placed in various positions to facilitate accurate positional and motion detection. Such markers are shown, e.g., in the lines and rectangular objects on the rig suit FIGS. 8A-8E and the lateral rods on the rig of FIGS. 1 and 3-7. In some embodiments, optical motion capture devices are used to capture the motion of the body. Such detection systems comprise passive markers, e.g., that deflect light, and active markers that emit light, e.g., LED light, infrared, or some other detectable signal. The active markers can be time modulated to facilitate accurate detection, e.g., by having different sensors emit light or other signal on a schedule. In some embodiments, no special markers are placed on the suit and the optical detection device directly focuses on the body alone. In still other embodiments, sensors placed in appropriate positions on rigs or suits of the invention transmit a signal indicative of their position. For example, inertial motion and/or oscillation sensors can transmit coordinates to a computer system. The transmission may be performed wirelessly to allow the subject's movement to be unencumbered by wiring. Similarly, magnetic sensors can be used to transmit motion and/or position information.

Functional envelopes are three dimensional polygons created by analysis of complex biomechanics of a subject. The polygons enable extrapolation of a biomechanical envelope of function (EOF) which can be used to identify a pattern with respect to movement of a joint, bone or skeletal structure, including without limitation the spine, neck, hip, knee, ankle, wrist, elbow, and/or shoulder. This pattern can be used both practically and theoretically. The functional mathematics established via a study of baselines EOFs can be used in relation to joint mechanics. For example, EOFs can be used to calculate functional singular and multi joint ranges of motion which can be created based on a theoretical biomechanical model and/or created based on a real time subject. Comparative and scalar functional analysis of EOFs can be performed. As will be appreciated by those skilled in the art, logic algorithms and software can be designed for use on an appropriate medium that gathers and transforms data associated with both macro and micro body movements. The movements can be detected using the motion capture rigs and suits as disclosed herein. The detected motion can then be converted with logic algorithms into EOF motion for analysis. In some embodiments, the EOF motion of the subject is compared to comparative EOF patterns determined by the logic system. In some cases, the subject's motion is compared to EOF patterns modeled in software to depict idealized motion, e.g., to detect motion error and determine a diagnosis. In other cases, the subject's motion is compared to the same subject's motion stored from other motion capture sessions, thereby to monitoring a treatment efficacy. In some cases, the motion is compared to the subject's motion captured in the same session, e.g., to compare natural motions to similar movements made with adjustments directed by the clinician. In still other cases, the subject's motion is compared to that of another subject, e.g., the subject's motion can be compared to that of a healthy person to provide a diagnosis or professional athlete to improve the subject's performance. These comparisons can allow the software to determine range of motion and biomechanical anomalies, dysfunctional system related soft tissue injury and performance or stress related ranges of motion.

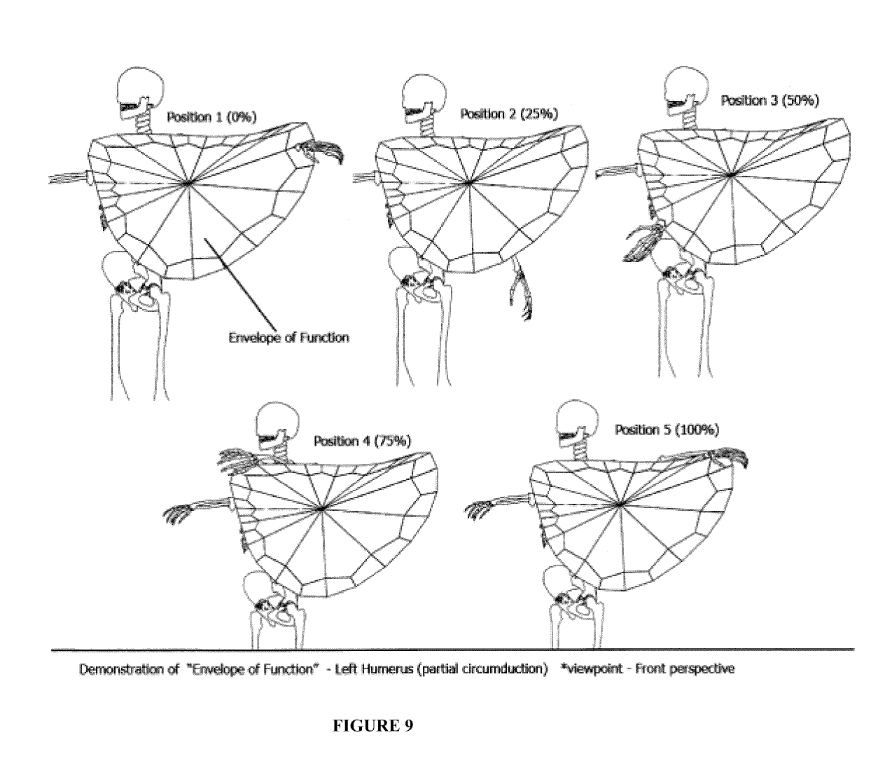

FIG. 9 illustrates a front perspective view showing envelopes of function. Position 1 shows the skeleton with the arm parallel to the ground and positioned perpendicular to the torso. Envelopes of function are shown around a central axis. Position 2 illustrates the subject dropping his arm toward his side. Position 3 illustrates the arm positioned forward of the torso, but still positioning the hands towards the hips. Position 4 illustrates the arms reaching forward, parallel to the ground. Position 5 returns the arm to the starting position of Position 1. For purposes of illustration, five positions are shown in FIG. 9. However, those of skill in the art will readily appreciate that more than five positions can be used to achieve greater granularity of the data. Each Position is represented by a range of motion shown in percentage. From the starting point of a motion, 0%, through a complete motion 100%.

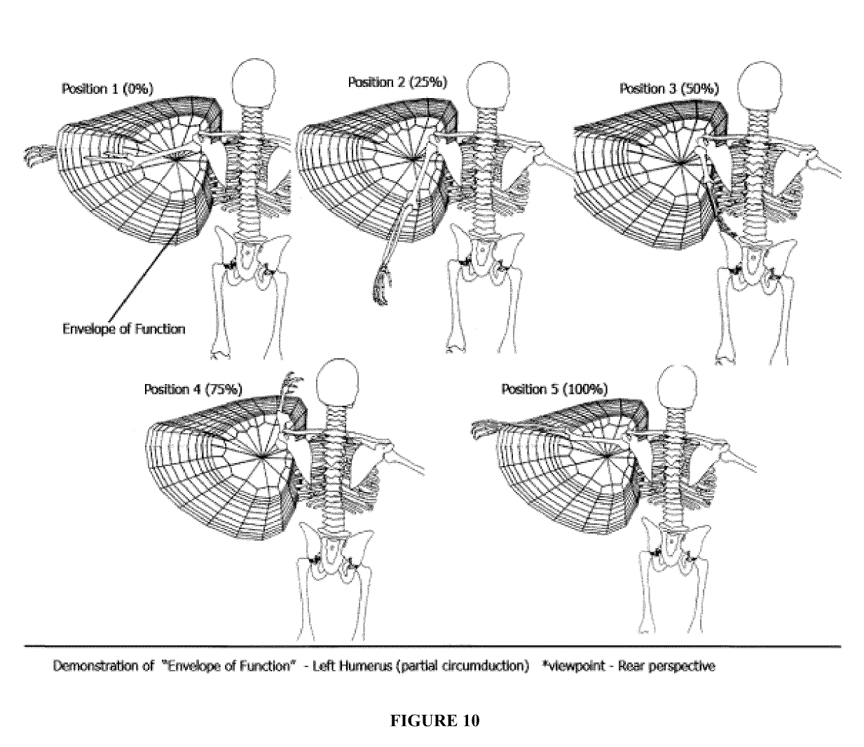

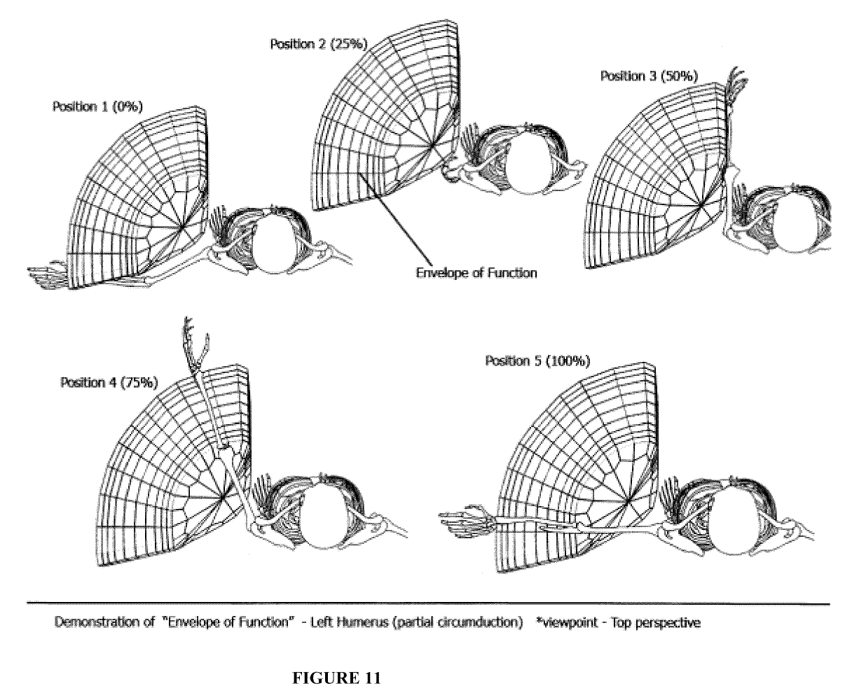

FIG. 10 illustrates a rear perspective view showing the envelopes of function through the same five positions as show in FIG. 9. FIG. 11 illustrates a top perspective view showing the envelopes of function through the same five positions shown in FIGS. 9 and 10. The webbed lines shown in the figures are extrapolated vertices. The more lines that are extrapolated, the tighter the geo poly design and thus the greater degree of accuracy achieved by the system. Thus, systems can be designed to achieve a desired level of accuracy by manipulating the geo poly design.

The envelopes of function enable data analysis that eliminates scalar values. Thus, whether a person is any height, e.g., 5 feet, 6 feet or 7 feet tall, the accuracy of the EOF data generated by motion capture of the will be substantially the same. As will be appreciated by those skilled in the art, every movement made by a subject can be interpreted relative to an x-y-z plane. Therefore, every bone in the subject's body functions in such a way that yaw, pitch and roll can be tracked through the x-y-z plane. Functionally, then each structure has its own gimbal system. A gimbal is a pivoted support that allows the rotation of an object about a single axis. Use of the EOF allows the creation of a volume polygon through trackable ranges of motion which enables an analysis of a yaw, pitch and roll for each structure that better tracks a movement, e.g., to identify motion defects. Sensors applied to a subject's body can be detected to enable the system to create a volume. The volume enables a real time extrapolation from motion sensors.

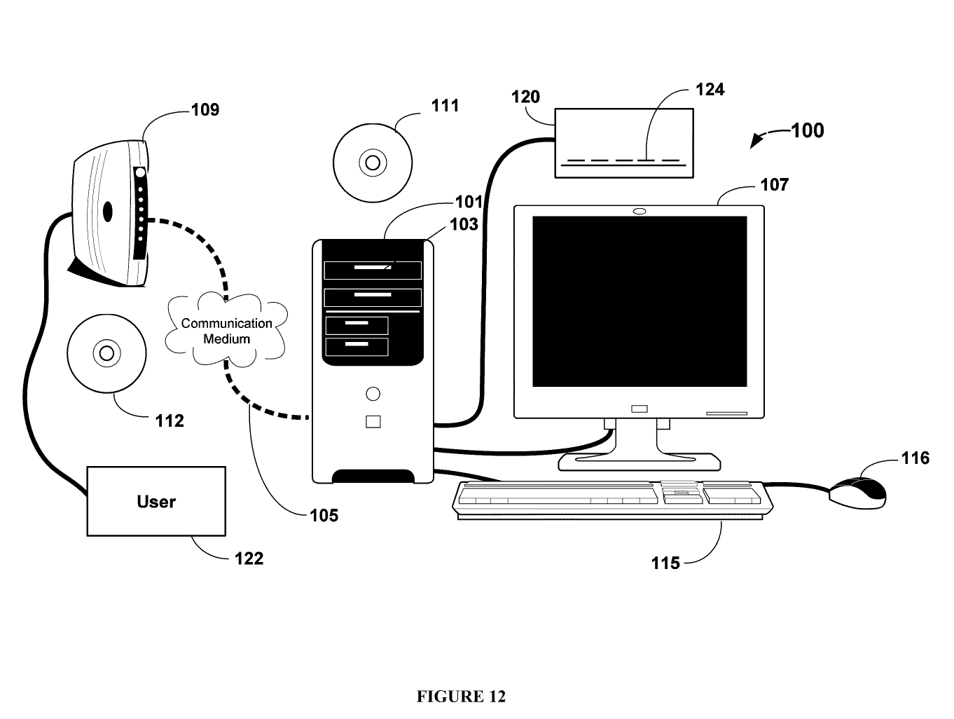

FIG. 12 is a diagram showing a representative example logic device through which reviewing or analyzing data relating to the present invention can be achieved. Such data can be in relation to a physiological parameter, or any other suitable parameter desired to be measured of a mammalian subject. A computer system (or digital device) 100 that may be understood as a logical apparatus that can read instructions from media 111 and/or network port 105, which can optionally be connected to server 109 having fixed media 112. The computer system 100 can also be connected to the Internet or an intranet using a wired or wireless connection. The system includes CPU 101, disk drives 103, optional input devices, illustrated as keyboard 115 and/or mouse 116 and optional monitor 107. Data communication can be achieved through the indicated communication medium to a server 109 at a local or a remote location. The communication medium can include any means of transmitting and/or receiving data. For example, the communication medium can be a network connection, a wireless connection or an internet connection. It is envisioned that data relating to the present invention can be transmitted over such networks or connections. The computer system can be adapted to communicate with a participant parameter monitor.

A user or participant 122 can also be connected to a variety of monitoring devices. The monitoring devices can be used to interact with the system. As will be appreciated by those skilled in the art, the computer system, or digital device, 100 can be any suitable device. In some embodiments, the subject's motion is tracked using a motion capture device of the invention and the motion is analyzed by EOF. In some embodiments, a subject is monitored by a motion capture device that monitors a joint, bone or skeletal structure, for example, the spine, neck, hip, knee, ankle, wrist, elbow, and/or shoulder. The motion is analyzed in terms of EOF and a computer system is used to analyze such motion. For example, the EOF of the subject can be compared to that of a computer generated ideal motion, e.g., the modeled motion of the subject without motion defects or the motion of a normal control subject, or a motion captured from the same or other subjects. Thus, the motion capture device of the invention works in concert with EOF analysis to provide an analysis of a subject's motion as described herein.

In an embodiment, the systems of the invention provide real-time in motion diagnostic information in 3D. The information can be displayed on a computer monitor 107 or similar display in a stand alone application or via a web-based system use a secure web server. The systems provide real-time, 3D biomechanical imagery captured by the motion capture equipment to the display device. The visualization can incorporate without limitation 3D medical anatomical displays, biomechanical data interpretation and interactive imaging that is needed for the diagnosis and/or treatment of the subject's body. In some embodiments, the computer systems incorporate a therapeutic solution that provides visual and verbal guidance instructions directing the steps needed for restoration of the injury based on the detected motion. For example, the system can compare the detected motion of the subject to an idealized motion, either modeled against a normal healthy movement or modeled against an ideal motion of the subject.

In embodiments, the system incorporates logic algorithms that can translate the data from the subjects' body into a computer generated muscular skeletal version, which can be scaled to size and made biomechanically accurate to medical standards. The muscular skeletal version can simulate joint function, joint movement, and muscle function, including without limitation active and passive muscle contractions, of agonists, synergists, antagonists and fixator muscle groups. When the subject moves while wearing one or more rigs or a full or partial motion capture suit, the muscular skeletal version can be duplicated by monitoring sensors that show what the subject is doing and how the body is accomplishing the motion by displaying the joint functions of the body. The use of the logic algorithms can help to identify dysfunctions, and can in many cases identify probable causes of the dysfunction. In some embodiments, the logic algorithms can visually and verbally guide the trainer, therapist, doctor or other care provider in step by step process to assist the subject body to reset its own dysfunctions, e.g., by using a procedure for resetting neuromuscular skeletal dysfunctions.

In some embodiments, the subject is monitored by the logic algorithms of the invention in one site and then captured movements are transmitted to an alternate site. The alternate site could have a server to store subject information. Analysts, therapists, sports medicine professionals or other service providers can be located at the alternate site to provide analysis and potentially recommendations for diagnosis and/or treatment. In some embodiments, the two sites are located in different physical locations, e.g., different rooms, wings, floors, buildings, neighborhoods, cities, states, countries or continents. Thus, the motion capture systems can be deployed in a single location or spread across multiple locations.

Backups of the collected subject data can be performed on a schedule, e.g., daily, every 2, 3, 4, 5, 6 days, or weekly.

### Motion Sensing Station

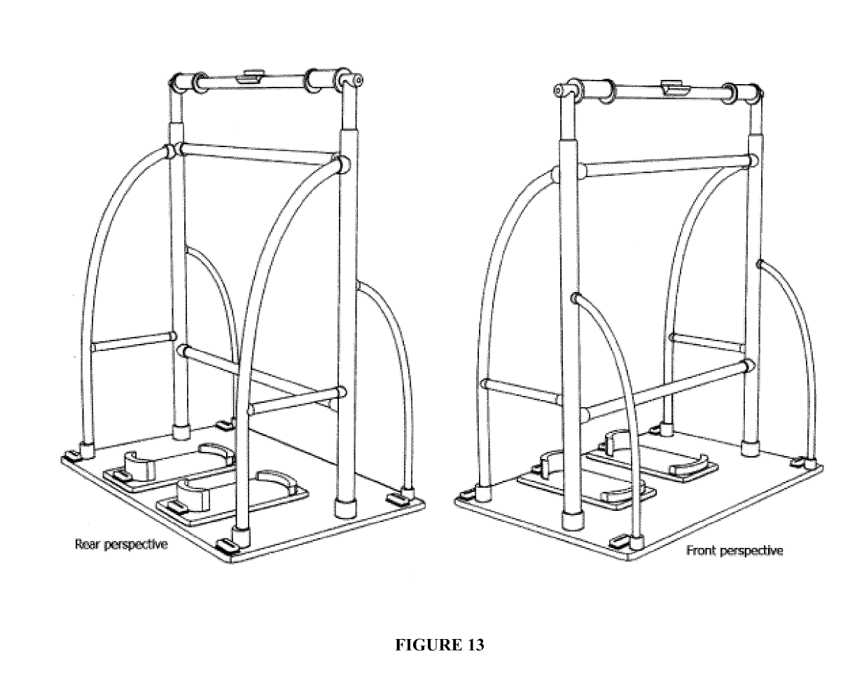

A motion sensing station is provided by the invention to further enable detection and analysis of a subject's motion. Such motion sensing station can be adapted to provide a pressure sensitive frame work that enables the subject to be placed into a position whereby the subject maintains a structural position, joint tension and balance. The station enables sensor placement to achieve a high degree of consistency. In most instances, this enables the sensor to achieve greater than 90% consistency and up to 100% consistency. In some embodiments, the consistency achieved via use of a motion sensing station is at least 70%, 75%, 80%, 85%, 90%, 91%, 92%, 93%, 94%, 95%, 96%, 97%, 98% or at least 99%. In embodiments, the framework for the motion sensing station consists of a platform adapted to include a pressure pad. The pressure pad is configured such that the subject can place all or part of their weight on the pad. One pressure pad may be provided for each foot or a single pressure pad may be provided that is configured to enable the subject to stand on the single pad. As depicted in FIGS. 13-16, right and left hand placement sections are provided which include another pressure pad. The station can be used together with the rigs provided herein. For example, a spinal sensor, spine rig, head sensor and/or head rig can be provided. The rig can be used to ensure position consistency. The station and motion sensing rig can be used in a system to monitor and analyze motion. In some embodiments, the subject wears a motion sensing suit as described herein while in position on the motion sensing station. Each of these elements, or combinations of the elements used together, will ensure that a subject is in the exact position each and every time the rig or suit is calibrated. This, in turn, increases the functional consistency achieved relative to a subject by different practitioners.

FIG. 13 illustrates a front and rear perspective view of a station according to the invention. The station shown is envisioned for a biped, e.g., a human, although similar configurations can be adapted for other subjects as described herein. The station consists of a frame work having static pressure sensitive areas. The station is configured to enable a subject, e.g., a human, to stand on a platform placing his or her feet within two foot receiving areas on the base. Side railings are provided. In some embodiments, a pressure sensitive rail is provided that enables a user to hold the rail at a position where the rail is adapted to sense the pressure exerted by the subject on the rail. Pressure sensors can be placed on the side rails as well.

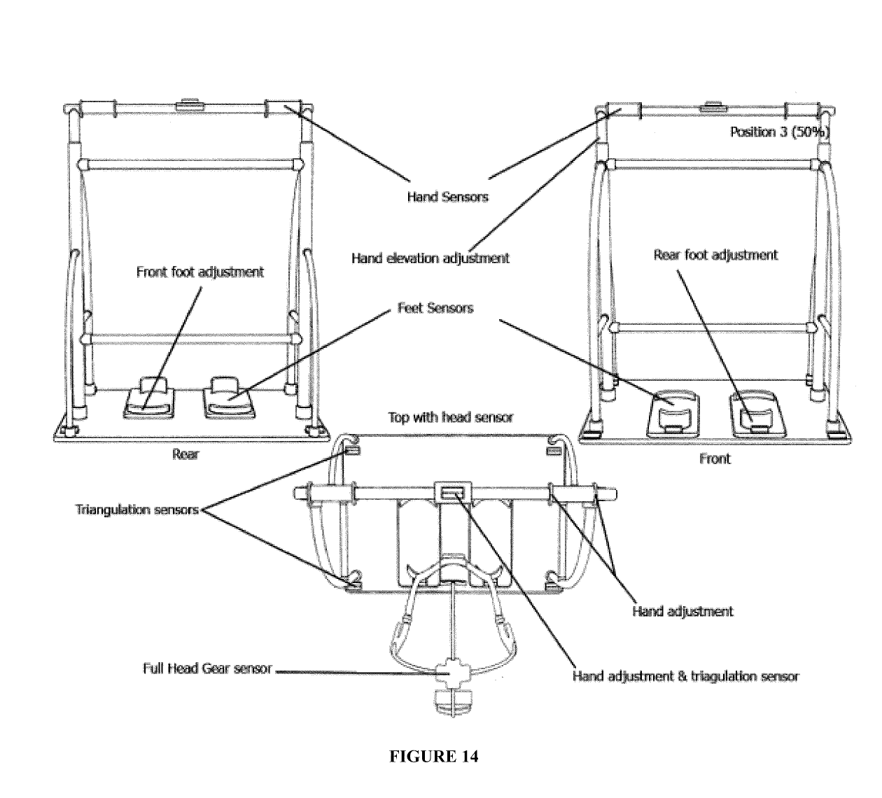

FIG. 14 illustrates front, rear and top views of a station according to the invention. As evident from this figure, the foot receiving areas can be configured such that they are adjustable to accommodate a variety of sizes. The adjustment can be achieved at one or both of the anteriorly or the posteriorly. Additionally, the side rails can enable the rail for hands to be adjusted vertically to accommodate subjects of different height. The hand sensors can also be adjustable. For example, where the hand sensors are provided on a rail that is positioned parallel or substantially parallel to the ground, the sensors can move horizontally together or separately along the rail. Where the hand sensors are provided on another rail, e.g. a side rail that curves toward the floor, the sensors may be moveable along those rails in a different orientation. Triangulation sensors are also provided.

One of skill will understand that the station can be configured in alternate shapes or positions, and can be adaptable in a variety of positions. For example, the hand sensors on the center rail can be configured to rotate in any direction and translate across any plane. Such adjustability can allow the user to be positioned into various defined positions or make movements while in the station. One of the skill will appreciate that the rotation and translation of the hand sensors would be limited by the physical motion of the subject. The rails can also be adjustable. In some embodiments, the center rail can move horizontally or vertically, thus allowing the subject to move, e.g., from straight standing position to a leaning, hunched or stretched position. The rails can be moved in a prescribed pattern while the subject is grasping the rails, thereby allowing the system to track the subject's biomechanical action while moving in a defined manner. Similarly, the footpads can be adjustable to position the subject in different positions. The base can also be adapted to move in prescribed manner to allow the system to track the subject's biomechanical action while moving in a defined manner.

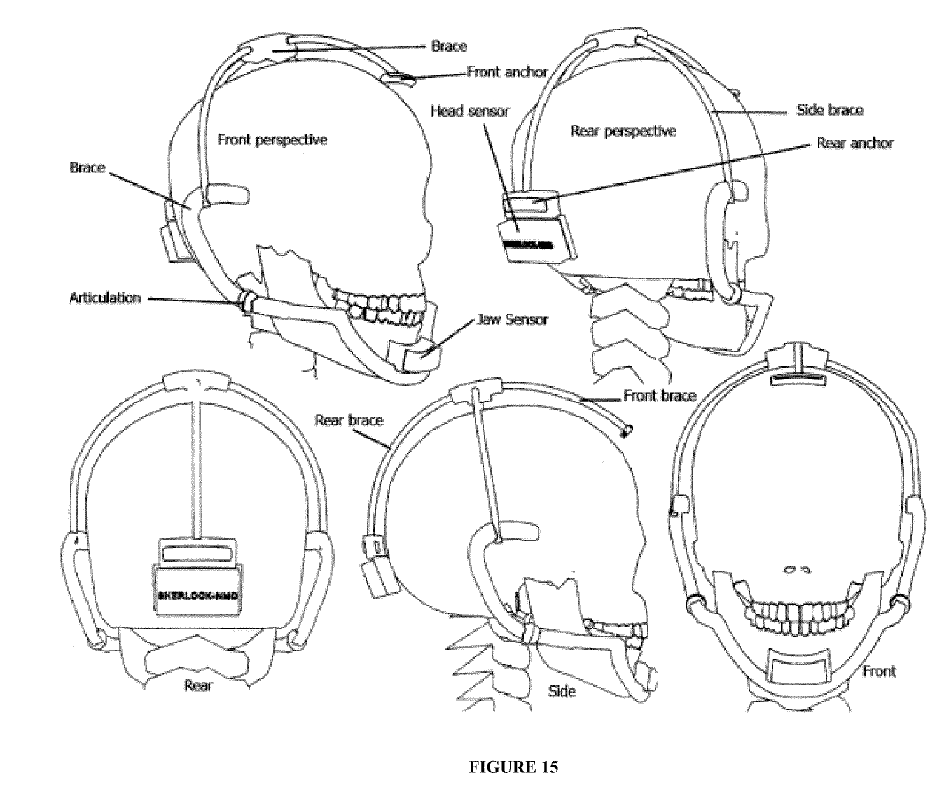

FIG. 15 illustrates a human skull from different perspectives having a head rig or the invention associated with it. The head rig has a front anchor, one or more braces, and a jaw sensor. It can be used to provide a superior, anterior, posterior and lateral anchor. In addition, the head rig motion capture duties can provide detailed yaw, pitch, and roll information in relation to head and cervical movement and dysfunction.

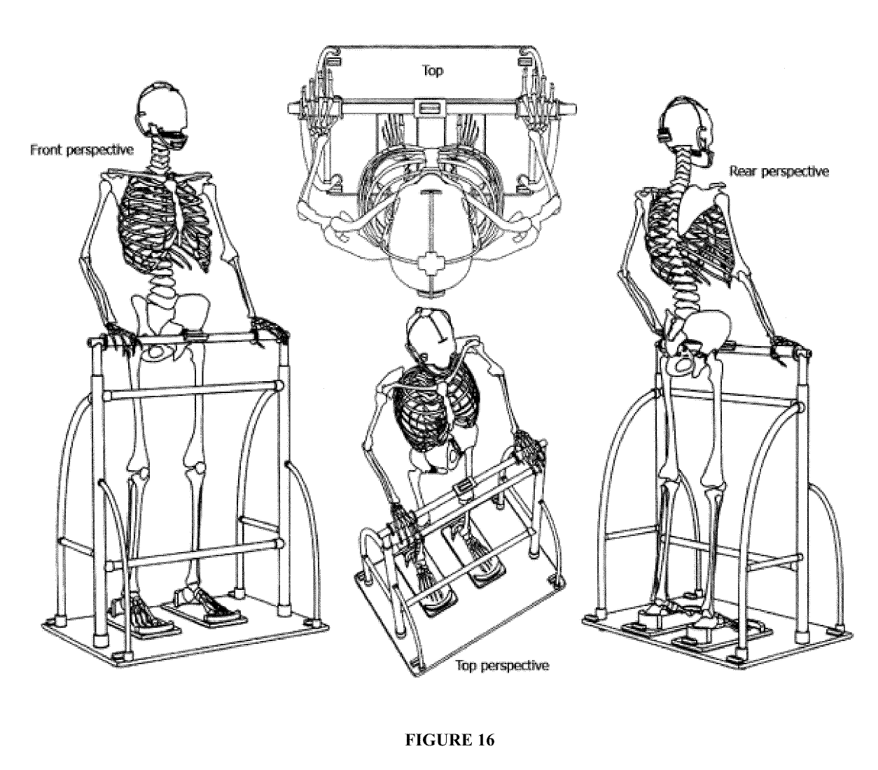

FIG. 16 illustrates a human skeleton with a head rig standing on a station. The station can provide a consistent environment to the deployment of the sensor array, e.g., to help eliminate continuity concerns between various practitioners. The sensor station can also provide a static environment, like a jig or mold used to create consistent copies of an implement. Thus, the sensor station can provide setup and configuration of the suit in relation to the human subject with no variation between different practitioners' setups. The sensor station can also be used to monitor the subject while in predefined positions.

Diagnostic Applications

The motion capture devices, systems and methods of the invention comprise one or more of diagnostic and therapeutic components. The diagnostic phase can involve the subject donning a rig as described herein or a special sensor suit that facilitates motion capture of the subject's movements in real time. As described, the captured motion information can be sent to a local or remote storage location, e.g., a database system. The therapeutic component can include artificial intelligence (A.I.) algorithms, e.g., to analyze the motion data and determine treatment protocols and instructions for these protocols. Exemplary diagnostic and therapeutic applications are described in more detail below. One of skill will appreciate that a number of modifications can be made without departing from the scope of the invention.

Preparatory Phase

Parameters of a rig or suit are adjusted to the subject and initial data is collected. The height, weight, body, three-dimensional distance between landmarks, appendage circumference and body fat of the subject are measured. These dimensions and other subject information, including without limitation the subject's physiological, congenital, surgical, pharmacological history, current signs/symptoms, current static imaging (example: MRI, CT, x-ray . . . ) and existing treatment protocols are entered into a database accessible by logic algorithms that capture and analyze the subject's motion. These parameters can be introduced to the subject's three-dimensional counterpart using various parametric inputs designed to mimic the subject's current existing musculoskeletal condition.

The suit is calibrated to the subject's primary sensory registry points and/or bone landmarks to facilitate stable analysis of functional biomechanics. Sensor positioning is calibrated based on various optical and accessory sensor implements locating landmarks and established biomechanical positions of reference. The landmarks and sensor positions are monitored in real time throughout the entire diagnostic. The suit can be adjusted with the aid of a sensor station as described here, to help provide setup and configuration of the suit in relation to the human subject with no variation between different practitioners' setups. The sensor station can be used to place the subject in a defined position as the sensors and suit are adjusted and calibrated.

The suit and logic software are also so that a functional relationship is determined between the sensors on the subject and the 3D version of the subject modeled in the logic software. Dimensional measurements via sensor communication can be calculated and verified against the input data of the actual physical measurements of the subject. This can help ensure a high degree of accuracy in measure the subject's motion. The system scales with subject, e.g., whether the subject is 140 lbs, 5′2″ and 7% body fat or 300 lbs, 6′6″ and 30% body fat, or any other height and weight that can be accommodated by the system.

Diagnostic error testing is performed to ensure medically accurate integration between the hardware and software components of the system. A baseline can be created as a starting point. This baseline starting point helps to ensure accuracy during multiple tests of the subject, e.g., over the course of a treatment. The time frames could vary from days to months or more. A new baseline can be created if there are any substantial changes in the subject's dimensions. Baselines can be monitored in real time for anomalies showing sensor misalignment to the subject and the subject's 3D counterpart.

Real-Time Biomechanical Monitoring Phase

The biomechanical monitoring can be recorded in 3D animation cycles for mathematical analysis by artificial intelligence (A.I.) monitoring by the logic systems and/or visual analysis by the subject's medical or training team.

Once accuracy is established, the subject can be monitored performing selected biomechanical movements designed to provide a meaningful picture of the functional biomechanics of the subject and/or possible anomalies in the subject's lack of biomechanical/functional range of motion (ROM).

Once motion data has been acquired, the logic software can analyze biomechanical ROM and may ask the subject to perform further movements and activities based on the correlative data and A.I. interpretation of the subject's ROM. At this point, the A.I. analysis can be performed in a preliminary mode in order to pick out any macro-anomalies in the subject's biomechanics.

After a preliminary evaluation, another dynamic phase of motion capture can be performed. For example, the subject might perform activities that have proven difficult or painful. The subject can indicate symptoms during these activities and the conducting medical team can document any verbal or visual indications of pain or other anomaly. Both signs and symptoms can be entered into the capturing device via verbal communication to ensure a fully immersed sense of cohesion between the subject and the logic software.

When using a remote or networked system to collect and/or analyze the motion capture data, the collected data and A.I. analysis can be uploaded to a server for storage. The data can be transferred in a secure manner, e.g., under encryption such as 128-bit encryption.

Throughout the process, the 3D content captured and/or modeled by the system can be reviewed through a graphic user interface (G.U.I.), e.g., on monitor 107. In embodiments, the system is adapted such that the subject information is viewable from unlimited viewpoints and unlimited levels of detail (from base skeletal, to the entire subject anatomy), both of which can be calculated by the logic algorithms.

Primary Analysis Phase

During the primary analysis phase, the A.I. components of the system may begin to derive an initial treatment protocol. In some embodiments, if there is anomalous data, the system may request further biomechanical monitoring or interactive monitoring. The rig or suit may also be adjusted or recalibrated, in some cases with the assistance of a motion sensing station. The subject and/or care giving team can view and analyze recorded or real time 3D data, or interact manually with the model, e.g., by changing viewing angle, zoom, speed, or other visual analysis components.

While the primary analysis phase is underway, which could take minutes or hours depending on the volume of data collected, further real-time visual analysis can be conducted. The primary analysis phase can use a combination of A.I. analysis (both visual and mathematical) and biomechanical references based on purely functional ROM (medically accepted human biomechanics and structural ROM).

Therapeutic Applications

The analysis of the subject's motion using the systems, devices and methods of the invention can be used to determine a treatment protocol. For example, upon conclusion of a diagnostic phase as described herein, the collected motion data can be used to determine treatment protocols, display visual variations in the subject's biomechanics and define treatment theories outlining the subject's neuromuscular diagnostics. During or after the motion capture stages, the logic algorithms can begin a detailed breakdown of protocols suggested for treatment of the subject's motion disorder. The subject can then be treated according to the suggestions, e.g., by undergoing physical therapy.

Based on the subject's treatment progress, further real-time biomechanical monitoring using the systems described herein can be performed. In some cases, one or more follow-up analysis sessions using the motion capture devices will be needed to determine a subject's progress. In some embodiments, the system calculates a % base improvement on the subject's functional biomechanics and signs/symptoms.

At any given time, the subject can be tested and placed back into the primary analysis diagnostic phase. In some embodiments, the A.I. database can take the new information and add it to the previous information to track the subject's progress. In other embodiments, a completely new file can be created on a previous subject. Data that is out dated can be either ignored or discarded from analysis.

Once the subject reaches a viable % of improvement, the can be used to provide maintenance recommendations in order to maintain neuromuscular health, e.g., based on the subject's entire file and current physical demands. In some cases, the systems calculate potential issues that may cause future neuromuscular dysfunction.

### EXAMPLES

### Example 1

### Medical Treatment of Muscular Skeletal Injury

Standard practice today for diagnosing muscular skeletal injuries includes the following:
Consultation—SOAP (subjective data, objective data, assessment and plan)
Radio diagnostic imaging studies
Laboratory studies
Treatment (conservative)
Rest, Medications, external support
Physiotherapy, occupational therapy, speech therapy etc
Surgical options (if conservative therapy fails)

Using the systems, devices and methods of the invention, diagnosis of such conditions can be performed as follows:
Consultation—(subjective data, objective data, assessment and plan)
Radio diagnostic imaging (studies)
MRI (at the option of the Medical personnel)
Noninvasive Diagnostic session using the motion capture systems and devices of the invention performed by a certified technician or similar care provider
The treating physician uses the results to discover if there are any biomechanical-musculoskeletal dysfunctions. The information is given to the physical therapist, chiropractor, orthopedist, etc to assist in creating an optimal treatment and follow-up therapy plan.
Surgical options (if conservative therapy fails)

### Example 2

### Sports Medicine Treatment

A trainer is working with a European football player, who keeps complaining that every time he kicks a soccer ball, he immediately feels a sharp pain in his hip, and then it goes away. The player is put through an X-ray and then an MRI, and neither shows abnormalities.

The player is troubled by the pain and without knowing, suddenly starts to change his kicking mechanics. The deterioration in his level of performance begins to show and the changes in his kicking mechanics have predisposed him to further problems. In most cases, the team will still try to play him injured, which in many cases, leads to career ending injuries.

The trainer uses the motion capture systems of the invention to further define the problem. The athlete puts on a motion capture suit (set up time is about 20 min), and then the trainer logs onto the secured web server and starts a file for the athlete. Next, the trainer has the athlete duplicate normal football moves such as a kick (strike). Every movement he makes is recorded and displayed in real time. After a few minutes of basic movement in the flexible suit, the trainer has the athlete review the results of the initial scanning.

It is played in slow motion to show the player what his body is doing. The trainer then asks the athlete at what point during the kick, does the pain occur. The athlete points out, “right there” and the analysis views are paused on the specific area.

The trainer then clicks on the hip joint; with each click a deeper layer of anatomy is shown. With four clicks the trainer moves through the layers of muscle and can now see the position of the actual joint. The trainer and athlete notice that the leg bone (femur) is jamming into the joint (acetabulum), most likely leading to the pain.

This is a great moment for an athlete who has an undiagnosed injury and answers the questions of why has there been so much pain and why he has been playing so poorly.

The A.I. program of the invention can then visually and verbally direct the trainer, step by step, muscle by muscle, how to help the athlete's body to reset its own dysfunctions. After resetting the player's dysfunctions, the athlete can put on the suit and allow the trainer to monitor the progress made by the treatment program.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device for capturing motion from a body comprising a rig adapted to conform to an external shape of the body, wherein the rig comprises two or more elongate members connected by two or more support members.

2. The device of claim 1, wherein the device is adapted to conform to at least a portion of a shape of an animal.

3. The device of claim 2, wherein the animal is a mammal, human, monkey, primate, horse, cow, dog, cat, rodent, guinea pig, rat or mouse.

4. The device of claim 1, wherein the device is adapted to conform to a joint, bone or skeletal structure of the body.

5. The device of claim 1, wherein the elongate members comprise a series of telescoping members.

6. The device of claim 5, wherein the telescoping functionality comprises a gas charging or liquid charging element.

7. The device of claim 1, further comprising one or more sensors in communication with the elongate members and/or support members.

8. The device of claim 7, wherein the sensors are in electrical communication with the elongate members and/or support members.

9. The device of claim 8, wherein the sensors are connected to the elongate members and/or support members via a damped universal joint.

10. The device of claim 7, wherein the sensors comprise at least one audio sensor, vibration sensor, or oscillation sensor.

11. The device of claim 7, wherein the sensors are adaptable to triangulate a plane of the body.

12. The device of claim 1, wherein the support members are connected to the elongate members by an axis ball socket, constant velocity or universal socket system.

13. The device of claim 1, wherein the device comprises at least two, three, four, five, six, seven, eight, nine or at least ten elongate members.

14. The device of claim 1, wherein the device comprises at least two, three, four, five, six, seven, eight, nine, 10, 11, 12, 13, 14, 15, 16, 17, 18, 19 or at least 20 support members.

15. The device of claim 1, wherein the rig is adapted to conform to a spine of the body.

16. The device of claim 15, wherein the device comprises three elongate members.

17. The device of claim 16, wherein the device comprises 5, 6, 7, 8, 9 or 10 support members.

18. The device of claim 15, wherein the device comprises a skull rig connected to the spine rig.

19. The device of claim 15, wherein the device further comprises a rig adapted to conform to one or more arms and/or one or more legs of the body.

20. The device of claim 1, wherein the rig is incorporated into an article of clothing adapted to be worn on the body.

21. The device of claim 20, wherein the article of clothing comprises a full or partial body suit.

22. The device of claim 20, wherein the article of clothing comprises an organic living exoskeletal morphometry.

23. The device of claim 21, wherein the body suit comprises an organic living exoskeletal morphometry and/or a flexible form fitting material.

24. The device of claim 23, wherein the flexible form fitting material comprises neoprene, nylon backed neoprene, lycra backed neoprene, cotton, nylon, polyester, elastene, or wool.

25. The device of claim 1, wherein the device is adapted to analyze motion captured from the body using envelopes of function.

26. The device of claim 25, wherein the device is adapted to track yaw, pitch and roll via the rig.

27. A system for capturing motion from a body comprising a device according to claim 1 and a computer system configured to capture and/or analyze motion of the body.

28. The system of claim 27, wherein the system is adapted to analyze the motion of the body using envelopes of function.

29. The system of claim 27, wherein the system is adapted to compare the motion of the body to a model of ideal motion.

30. A method for capturing motion from a body comprising:
(a) providing a device according to claim 1;
(b) conforming the rig to the body; and
(c) capturing the motion of the body using the rig.

31. The method of claim 30, wherein step (b) comprises attaching one or more sensors to triangulated positions relative to bony landmarks in the body and/or structural dead areas of the body.

32. The method of claim 30, further comprising analyzing motion data from the body using envelopes of function.

33. The method of claim 30, further comprising comparing the motion of the body to an ideal motion.

34. The method of claim 33, wherein the comparison is used to diagnose a motion disorder or determine the efficacy of a course of treatment for treating a motion disorder.

35. A method for capturing motion from a body comprising:
(a) providing a system according to claim 27;
(b) conforming the rig to the body; and
(c) capturing the motion of the body using the rig.

36. The method of claim 35, wherein step (b) comprises attaching one or more sensors to triangulated positions relative to bony landmarks in the body and/or structural dead areas of the body.

37. The method of claim 35, further comprising analyzing motion data from the body using envelopes of function.

38. The method of claim 35, further comprising comparing the motion of the body to an ideal motion.

39. The method of claim 38, wherein the comparison is used to diagnose a motion disorder or to determine the efficacy of a course of treatment for treating a motion disorder.

40. An adjustable station adapted to capture a sensed parameter from a body, the station comprising a base plate and a support framework protruding from the base plate, wherein the support framework comprises a support rail.

41. The device of claim 40, wherein the base plate is substantially flat.

42. The device of claim 40, wherein the base plate comprises one or more pressure pads adapted to support a weight of the body.

43. The device of claim 42, wherein the pressure pads are adjustable to accommodate a variety of body sizes.

44. The device of claim 42, wherein the pressure pads are adjustable anteriorly and/or posteriorly.

45. The device of claim 40, wherein the support rail is adapted to be held onto by the body.

46. The device of claim 45, wherein the support framework comprises at least one side rail connected to the base plate, and wherein at least one of the at least one side rails support the support rail.

47. The device of claim 45, wherein the support rail is substantially perpendicular to the base plate.

48. The device of claim 45, wherein the support rail is vertically adjustable.

49. The device of claim 40, wherein the support framework comprises pressure sensors.

50. The device of claim 49, wherein the support framework pressure sensors are adapted to sense the pressure exerted by the body on the support rail.

51. The device of claim 40, wherein the support rail comprises one or more hand sensors that are adjustable along a length of the support rail.

52. The device of claim 46, wherein the one or more of the one or more side rails comprises a hand sensor that is adjustable along a length of the side rail.

53. The device of claim 40, wherein the adjustable station is adapted to capture a sensed parameter from a human body in a standing or crouched position.

54. A system comprising:
(a) a device according to any of claim 1; and
(b) an adjustable station according to claim 40.

55. The system of claim 54, further comprising a computer system configured to capture and/or analyze a position or a motion of the body.

56. The system of claim 54, wherein the system is adapted to analyze the motion of the body using envelopes of function.

57. The system of claim 54, wherein the system is adapted to compare a position or motion of the body to a model position or motion.

58. A method for calibrating a motion capture device placed on a body comprising:
(a) providing a device according to claim 1;
(b) providing an adjustable station according to claim 40; and
(c) conforming the device of step (a) to the body;
(d) placing the body on the base plate of the adjustable station and optionally having the body grasp the support rail of the adjustable station; and
(e) calibrating the device of step (a) to the body while the body is positioned in the adjustable station.

59. The method of claim 58, further comprising providing a computer system configured to capture and/or analyze a position and/or a motion of the body.

60. A method for diagnosing a muscular skeletal condition of a human subject, comprising:
(a) providing a flexible form fitting body suit adaptable to be worn by the subject, wherein the body suit comprises a series of sensors placed on the skull and placed along a length of the arms, legs, spine, and stomach areas of the body suit;
(b) providing an adjustable station comprising:
a base plate comprising two pressure sensing plates adapted to support the weight of the subject; and
a support framework protruding from the base plate, wherein the support framework comprises a support rail supported by two side rails, wherein the support rail is adapted to be held by the subject, and wherein the support rail comprises two adjustable hand sensors;
(c) providing a computer system configured to capture and/or analyze a position and/or a motion of the subject;
(d) having the subject don body suit;
(e) placing the subject in a standing position within the adjustable station with one foot positioned on one of the two pressure sensing plates, the other foot positioned on the other of the two pressure sensing plates, one hand holding one of the adjustable hand sensors on the support rail, and the other hand holding the other adjustable hand sensor on the support rail;
(f) adjusting the suit while the subject is standing within the adjustable station, wherein the adjusting comprises:
(i) comparing the position of the user and the sensors on the body suit against a 3D model of the user generated by the computer system; and
(ii) repositioning the suit and/or calibrating the detection system until the position of the user and the sensors on the body suit meet a desired level of calibration as determined by the computer system;
(g) capturing the motion of the subject while the subject is wearing the adjusted suit;
(h) transmitting the capture data to the computer system in real time;
(i) comparing the motion of the subject to a model of the same motion generated by the computer system; and
(j) using the comparison in step (h) to diagnose the muscular skeletal condition.

61. The method of claim 60, wherein the comparison in step (i) comprises analyzing the motion of the subject using envelopes of function.
